(19) United States
(12) Patent Application Publication
(10) Pub. No.: US 2022/0347289 A1
(43) Pub. Date: Nov. 3, 2022

(54) NUCLEOTIDE SEQUENCES ENCODING PEPTIDE ANTIGENS OF SARS-COV-2 VIRUS AND USE THEREOF

(71) Applicant: GUANGZHOU N BIOMED LTD., Guangzhou, Guangdong (CN)

(72) Inventors: Ling Chen, Guangzhou, Guangdong (CN); Suhua Guan, Guangzhou, Guangdong (CN); Chenchen Yang, Guangzhou, Guangdong (CN); Xiaolin Liu, Guangzhou, Guangdong (CN)

(21) Appl. No.: 17/260,160

(22) PCT Filed: Aug. 19, 2020
(86) PCT No.: PCT/CN2020/110086

§ 371 Date: Jan. 13, 2021

(30) Foreign Application Priority Data
Feb. 23, 2020 (CN) 202010110070.8
Mar. 5, 2020 (CN) 202010145657.2

(51) Int. Cl. A61K 39/215; A61P 31/14; C12N 15/86

(52) U.S. Cl. CPC A61K 39/215; A61P 31/14; C12N 15/86; A61K 2039/53

(57) ABSTRACT

The present disclosure discloses nucleotide sequences as shown by SEQ ID NO: 1, SEQ ID NO: 2 and the nucleotide sequences having at least 90% homology therewith, which encode peptide antigens of SARS-CoV-2 virus. According to some embodiments of the present disclosure, the nucleotide sequences can be effectively expressed in human cells and produce the corresponding peptides, thereby inducing an immune-protective response accordingly. Thus, the nucleotide sequences of the present disclosure are expected to be developed as a vaccine against SARS-CoV-2 infection.

### CROSS-REFERENCE TO RELATED APPLICATIONS

This application is based on and claims the benefit of priority from Chinese Patent Application No. 202010110070.8, filed on 23 Feb. 2020 and Chinese Patent Application No. 202010145657.2, filed on 5 Mar. 2020, the entirety of which is incorporated by reference herein.

### INCORPORATION BY REFERENCE

This application includes a sequence listing in computer readable form (a “txt” file) that is submitted herewith. This sequence listing is incorporated by reference herein.

### TECHNICAL FIELD

The present disclosure relates to an optimized coronavirus S protein and the use thereof, particularly to nucleotide sequences encoding peptide antigens of SARS-CoV-2 virus and the use thereof.

### BACKGROUND

Novel Severe Acute Respiratory Syndrome Coronavirus 2 (SARS-CoV-2) has a latent period up to 24 days, and is highly infectious. Unlike the virus that caused Severe Acute Respiratory Syndrome (SARS), some cases infected with SARS-CoV-2 have infectivity during the latent period, while some SARS-CoV-2 carriers do not show any obvious symptoms. This increases the difficulty of epidemic prevention and control. As of 20 Jul. 2020, there have been close to 15 million confirmed cases, including deaths of above 600,000, all over the world. So far there is no specific medicine available for coronavirus disease 2019 (COVID-19). To control the epidemic, most countries mandated quarantine and closed unnecessary facilities, which resulted in huge economic loss. Therefore, it has become the most urgent and major need to rapidly develop preventive vaccines that can improve herd-immunity level and block the transmission of the virus.

Currently, there is no specific antiviral medicine and preventive vaccine against this novel coronavirus, in China and other countries. Preventing and blocking the viral transmission are the keys to control the epidemic. It is known that vaccines have played an irreplaceable role in eliminating various infectious diseases. The published results obtained from the genome alignment of a dozen of SARS-CoV-2 viruses showed that the difference among the viruses is very small, and no mutation has been found. Therefore, the outbreak of new epidemic would be greatly contained, if SARS-CoV-2 vaccines are successfully developed.

SARS-CoV-2 has a complete genome as shown by NC 045512.2, in which the sequence of nucleic acids 21563 to 25384 is a coding sequence for Spike protein (S). The spike (S) protein is a major structural protein of a virion, and plays an important role in mediating the binding of the virion to a host cell receptor and inducing neutralizing antibodies. This makes the S protein an ideal vaccine antigen. The S protein comprises two subunits, S1 and S2. When the virus infects, the S1 subunit binds to the receptor, angiotensin-converting enzyme 2 (ACE2), on host cells. Then the S protein is cleaved by proteases found in the host cells, after which the 51 and S2 subunits are separated to accelerate the fusion of the S2 subunit with cell membrane. Vaccines, which use the S protein as an antigen, comprise nucleic acid-, subunit- and virus-vectored vaccines, and the efficacy of such vaccines are determined by the expression level and protein structure of the S protein.

However, it was shown by experiments that the gene coding for the Spike protein of natural SARS-CoV-2 has low expression level in the cells, such as Human embryonic kidney (HEK) 293 cells, commonly used for expressing subunit-vaccines. If a vaccine adopts natural genetic codons to express the S protein as the antigen, this vaccine would be ineffective or have low titer, thereby being unable to fight the virus infection.

### SUMMARY

The present disclosure is intended to provide a nucleotide sequence encoding a peptide which triggers immunogenicity against SARS-CoV-2, as well as the use thereof.

The technical solutions adopted by the present disclosure are as follows.

In a first aspect of the present disclosure, provided is a nucleotide sequence encoding a peptide which triggers immunogenicity against Novel Severe Acute Respiratory Syndrome Coronavirus 2 (SARS-CoV-2), which comprises at least one selected from:

a) a nucleotide sequence as shown by SEQ ID NO: 1; or

b) a nucleotide sequence as shown by SEQ ID NO: 2; or

c) a nucleotide sequence having at least 90%, preferably 95%, 98% or 99% sequence identity with the nucleotide sequence as shown by SEQ ID NO: 1.

In some embodiments, said peptide is capable of:

inducing an immune response in a human body;

generating a biology reporter;

generating a molecule for detection;

regulating a gene function; or

functioning as a therapeutic molecule.

In a second aspect of the present disclosure, provided is an expression vector which comprises the nucleotide sequence according to the first aspect of the present disclosure.

In some embodiments, the vector may be a viral vector.

In some embodiments, the viral vector may be an adenovirus vector.

In a third aspect of the present disclosure, provided is a cell expressing the peptide based on the nucleotide sequence according to the first aspect of the present disclosure.

In a fourth aspect of the present disclosure, provided is a nucleotide composition which comprises the nucleotide sequence according to the first aspect of the present disclosure, or the expression vector according to the second aspect of the present disclosure.

In some embodiments, the nucleotide composition may further comprise at least one of a pharmaceutically-acceptable adjuvant, carrier, diluent or excipient.

In a fifth aspect of the present disclosure, provided is use of the nucleotide composition according to the fourth aspect of the present disclosure in the preparation of an agent for preventing or treating COVID-19.

In a sixth aspect of the present disclosure, provided is a method for preventing or treating COVID-19, which comprises administering a prophylactically or therapeutically effective amount of the nucleotide composition according to the fourth aspect of the present disclosure to a human.

The beneficial effects of the present disclosure are as follows.

The nucleotide sequences according to some embodiments of the present disclosure can be effectively expressed in mammal cells and produce the corresponding peptides, thereby inducing an immune-protective response accordingly. Such nucleotide sequences are expected to be developed as a vaccine against SARS-CoV-2 infection.

### BRIEF DESCRIPTION OF THE DRAWINGS

FIG. 1 shows the expression of S protein after transfecting with a same dose of pVAX-NB1, pVAX-NB2 and pVAX-NB4.

FIG. 2 shows the antibody levels in serum of the mice immunized with pVAX-NB1- and pVAX-NB2-DNA vaccines.

### DETAILED DESCRIPTION

The Spike protein (S) of SARS-CoV-2 has an amino acid sequence as shown by YP 009724390.1, and the natural nucleotide sequence encoding the S protein of SARS-CoV-2 is designated as NB1.

Precursor mRNA in eukaryotic cells can produce various mRNA splice variants through different splicing manners (by selecting different combination of splice sites), eventually leading to different proteins produced from the same gene sequence. This is very unfavorable for the expression of a protein. The inventor performed codon optimization to the wild-type natural nucleotide sequence and removed potential variable cleavage sites based on self-owned technology, thereby ensuring the uniqueness of expressed protein and reducing the difficulty in subsequent protein purification. The optimized nucleotide sequence is designated as NB2, and is as shown by SEQ ID NO: 1:

(SEQ ID NO.: 1)
ATGTTCGTGTTTCTGGTGCTGCTGCCTCTGGTGAGCTCCCAGTGCGTGA
ACCTGACCACAAGGACCCAGCTGCCACCTGCCTATACCAATAGCTTCAC
ACGGGGCGTGTACTATCCCGACAAGGTGTTTAGATCTAGCGTGCTGCAC
TCCACCCAGGATCTGTTTCTGCCTTTCTTTTCTAACGTGACATGGTTCC
ACGCCATCCACGTGTCCGGCACCAATGGCACAAAGCGGTTCGACAATCC
AGTGCTGCCCTTTAACGATGGCGTGTACTTCGCCTCCACCGAGAAGTCT
AACATCATCAGAGGCTGGATCTTTGGCACCACACTGGACAGCAAGACCC
AGTCCCTGCTGATCGTGAACAATGCCACAAACGTGGTCATCAAGGTGTG
CGAGTTCCAGTTTTGTAATGATCCCTTCCTGGGCGTGTACTATCACAAG
AACAATAAGTCTTGGATGGAGAGCGAGTTTAGGGTGTATTCCTCTGCCA
ACAATTGCACCTTTGAGTACGTGAGCCAGCCTTTCCTGATGGACCTGGA
GGGCAAGCAGGGCAATTTCAAGAACCTGAGGGAGTTCGTGTTTAAGAAT
ATCGATGGCTACTTCAAGATCTACTCCAAGCACACACCAATCAACCTGG
TGCGCGACCTGCCACAGGGCTTCTCTGCCCTGGAGCCACTGGTGGATCT
GCCCATCGGCATCAACATCACCCGGTTTCAGACACTGCTGGCCCTGCAC
AGAAGCTACCTGACCCCAGGCGACAGCTCCTCTGGATGGACAGCAGGAG
CTGCCGCCTACTATGTGGGCTATCTGCAGCCCCGCACCTTCCTGCTGAA
GTACAACGAGAATGGCACCATCACAGACGCAGTGGATTGCGCCCTGGAC
CCCCTGTCTGAGACCAAGTGTACACTGAAGAGCTTTACAGTGGAGAAGG
GCATCTACCAGACCAGCAACTTCAGGGTGCAGCCAACAGAGTCCATCGT
GCGCTTTCCCAATATCACCAACCTGTGCCCTTTTGGCGAGGTGTTCAAT
GCCACACGCTTCGCCAGCGTGTACGCCTGGAATAGGAAGCGCATCTCCA
ACTGCGTGGCCGACTATTCTGTGCTGTACAACAGCGCCTCCTTCTCTAC
CTTTAAGTGTTATGGCGTGAGCCCCACCAAGCTGAATGATCTGTGCTTT
ACAAACGTGTACGCCGATTCCTTCGTGATCAGGGGCGACGAGGTGCGCC
AGATCGCACCAGGACAGACCGGCAAGATCGCAGACTACAATTATAAGCT
GCCTGACGATTTCACAGGCTGCGTGATCGCCTGGAACTCTAACAATCTG
GATAGCAAAGTGGGCGGCAACTACAATTATCTGTACCGGCTGTTTAGAA
AGTCTAATCTGAAGCCATTCGAGCGGGACATCTCCACCGAGATCTACCA
GGCCGGCTCTACACCCTGCAATGGCGTGGAGGGCTTTAACTGTTATTTC
CCTCTGCAGTCCTACGGCTTCCAGCCAACCAACGGCGTGGGCTATCAGC
CCTACAGAGTGGTGGTGCTGTCTTTTGAGCTGCTGCACGCACCTGCAAC
CGTGTGCGGCCCAAAGAAGAGCACAAATCTGGTGAAGAACAAGTGCGTG
AACTTCAACTTCAACGGACTGACCGGCACAGGCGTGCTGACCGAGAGCA
ACAAGAAGTTCCTGCCATTTCAGCAGTTCGGCAGGGACATCGCAGATAC
CACAGACGCCGTGCGCGACCCTCAGACCCTGGAGATCCTGGACATCACA
CCATGTTCCTTCGGCGGCGTGTCTGTGATCACCCCAGGCACCAATACAT
CCAACCAGGTGGCCGTGCTGTATCAGGACGTGAATTGCACAGAGGTGCC
CGTGGCAATCCACGCAGATCAGCTGACCCCTACATGGCGGGTGTACTCT
ACCGGCAGCAACGTGTTCCAGACAAGAGCCGGATGCCTGATCGGAGCAG
AGCACGTGAACAATAGCTATGAGTGCGACATCCCTATCGGCGCCGGCAT
CTGTGCCTCCTACCAGACCCAGACAAACTCCCCAAGGAGAGCCCGGTCT
GTGGCCAGCCAGTCCATCATCGCCTATACCATGAGCCTGGGCGCCGAGA
ACAGCGTGGCCTACTCCAACAATTCTATCGCCATCCCTACCAACTTCAC
AATCAGCGTGACCACAGAGATCCTGCCAGTGAGCATGACCAAGACATCC
GTGGACTGCACCATGTATATCTGTGGCGATTCCACAGAGTGTTCTAACC
TGCTGCTGCAGTACGGCTCCTTTTGCACCCAGCTGAATAGAGCCCTGAC
AGGCATCGCCGTGGAGCAGGACAAGAACACCCAGGAGGTGTTCGCCCAG
GTGAAGCAGATCTACAAGACACCACCCATCAAGGACTTTGGCGGCTTCA
ACTTCAGCCAGATCCTGCCCGATCCTAGCAAGCCATCCAAGCGGTCTTT
TATCGAGGACCTGCTGTTCAACAAGGTGACCCTGGCCGATGCCGGCTTC
ATCAAGCAGTATGGCGATTGTCTGGGCGACATCGCCGCCAGAGACCTGA
TCTGCGCCCAGAAGTTTAATGGCCTGACCGTGCTGCCTCCACTGCTGAC
AGATGAGATGATCGCACAGTACACCTCTGCCCTGCTGGCCGGCACCATC
ACAAGCGGATGGACATTCGGCGCAGGAGCCGCCCTGCAGATCCCCTTTG
CCATGCAGATGGCCTATCGGTTCAACGGCATCGGCGTGACCCAGAATGT
GCTGTACGAGAACCAGAAGCTGATCGCCAATCAGTTTAACAGCGCCATC
GGCAAGATCCAGGACTCTCTGAGCTCCACCGCCAGCGCCCTGGGCAAGC
TGCAGGATGTGGTGAATCAGAACGCCCAGGCCCTGAATACACTGGTGAA
GCAGCTGTCTAGCAACTTCGGCGCCATCTCCTCTGTGCTGAATGACATC
CTGAGCCGGCTGGACAAGGTGGAGGCAGAGGTGCAGATCGACCGGCTGA
TCACCGGCAGACTGCAGTCCCTGCAGACCTACGTGACACAGCAGCTGAT
CAGGGCAGCAGAGATCAGGGCCTCTGCCAATCTGGCCGCCACAAAGATG
AGCGAGTGCGTGCTGGGACAGTCCAAGAGGGTGGACTTTTGCGGCAAGG
GCTATCACCTGATGAGCTTCCCACAGTCCGCCCCTCACGGAGTGGTGTT
TCTGCACGTGACCTACGTGCCAGCCCAGGAGAAGAACTTCACCACAGCC
CCCGCCATCTGTCACGATGGCAAGGCCCACTTTCCTAGGGAGGGCGTGT
TCGTGAGCAACGGCACCCACTGGTTTGTGACACAGCGCAATTTCTACGA
GCCACAGATCATCACCACAGACAATACCTTCGTGTCCGGCAACTGCGAC
GTGGTCATCGGCATCGTGAACAATACAGTGTATGATCCTCTGCAGCCAG
AGCTGGACTCTTTTAAGGAGGAGCTGGATAAGTACTTCAAGAATCACAC
CAGCCCCGACGTGGATCTGGGCGACATCTCTGGCATCAATGCCAGCGTG
GTGAACATCCAGAAGGAGATCGACAGACTGAACGAGGTGGCCAAGAATC
TGAACGAGAGCCTGATCGATCTGCAGGAGCTGGGCAAGTATGAGCAGTA
CATCAAGTGGCCCTGGTATATCTGGCTGGGCTTCATCGCCGGCCTGATC
GCCATCGTGATGGTGACCATCATGCTGTGCTGTATGACAAGCTGCTGTT
CCTGCCTGAAGGGCTGCTGTTCTTGTGGCAGCTGCTGTAAGTTTGATGA
GGACGATTCCGAGCCTGTGCTGAAGGGCGTGAAGCTGCACTACACCTA
A.

In order to reduce the GC-content of the gene, the inventor further optimized the codons based on NB2, resulting in a sequence designated as NB4. NB4 has a sequence as shown by SEQ ID No.: 2:

(SEQ ID NO: 2)
ATGTTTGTCTTCCTGGTGCTGCTGCCTCTGGTCTCTTCTCAGTGTGTGA
ACCTGACCACCAGGACACAGCTGCCTCCTGCTTACACCAACTCCTTCAC
ACGGGGCGTCTACTACCCTGACAAGGTCTTCAGGTCCTCTGTGCTGCAC
TCCACACAAGACCTGTTCCTGCCATTCTTCTCCAACGTGACCTGGTTCC
ATGCCATCCATGTCTCTGGCACCAATGGCACCAAGAGGTTCGACAACCC
TGTGCTGCCATTCAACGACGGCGTCTACTTTGCCTCCACCGAGAAGTCC
AACATCATCAGGGGCTGGATCTTTGGCACCACCCTGGACTCCAAGACCC
AGTCCCTGCTGATTGTGAACAATGCCACCAACGTGGTGATCAAAGTCTG
CGAGTTCCAGTTCTGCAATGACCCATTCCTGGGCGTCTACTACCACAAG
AACAACAAGTCCTGGATGGAGTCTGAGTTCAGGGTCTACTCCTCTGCCA
ACAACTGCACCTTTGAATATGTCTCCCAGCCATTCCTGATGGACCTGGA
GGGCAAGCAGGGCAACTTCAAGAACCTGAGGGAGTTCGTCTTCAAGAAC
ATCGACGGCTACTTCAAGATCTACTCCAAGCACACACCCATTAATCTCG
TCCGGGATCTGCCTCAGGGCTTCTCTGCCCTGGAGCCCCTGGTGGACCT
GCCCATTGGCATCAACATCACACGCTTTCAGACCCTGCTGGCTCTGCAC
CGGTCTTACCTGACACCTGGCGACTCCTCCTCTGGCTGGACAGCTGGCG
CTGCTGCCTACTATGTCGGCTACCTGCAGCCTAGGACCTTCCTGCTGAA
GTACAATGAGAATGGCACCATCACAGATGCTGTGGACTGTGCCCTGGAC
CCCCTGTCTGAGACCAAGTGCACCCTGAAGTCCTTCACAGTCGAGAAGG
GCATCTACCAGACCTCCAACTTCCGGGTGCAGCCCACAGAGTCCATTGT
CAGGTTCCCCAACATCACCAACCTGTGCCCATTTGGCGAGGTCTTCAAT
GCCACACGCTTTGCCTCTGTCTATGCCTGGAACAGGAAGAGGATCTCCA
ACTGTGTGGCTGACTACTCTGTGCTGTACAACTCTGCCTCCTTCTCCAC
ATTTAAGTGCTATGGCGTCTCCCCCACCAAGCTGAATGACCTGTGCTTC
ACCAATGTCTATGCTGACTCCTTTGTGATCCGGGGCGATGAAGTTAGGC
AGATTGCCCCTGGCCAGACAGGCAAGATTGCTGACTACAACTACAAGCT
GCCTGATGACTTCACCGGCTGTGTGATTGCCTGGAACTCCAACAACCTG
GACTCTAAAGTTGGCGGCAACTACAACTACCTGTACAGGCTGTTCAGGA
AGTCCAACCTGAAGCCATTTGAGAGGGACATCTCCACAGAGATCTACCA
GGCTGGCTCCACCCCATGCAATGGCGTGGAGGGCTTCAACTGCTACTTC
CCCCTGCAATCCTATGGCTTCCAACCCACCAATGGCGTGGGCTACCAGC
CATACAGGGTGGTGGTGCTGTCCTTTGAGCTGCTGCATGCCCCTGCCAC
CGTCTGTGGCCCCAAGAAGTCCACCAACCTCGTGAAGAACAAATGTGTG
AACTTCAACTTCAACGGCCTGACAGGCACAGGCGTGCTGACAGAGTCCA
ACAAGAAGTTCCTGCCATTCCAACAGTTTGGCAGGGACATTGCTGACAC
CACCGATGCTGTCAGGGACCCCCAGACCCTGGAGATCCTGGACATCACC
CCATGCTCCTTTGGCGGCGTCTCTGTGATCACCCCTGGCACCAACACCT
CTAATCAAGTCGCTGTGCTGTACCAGGACGTGAACTGCACAGAGGTTCC
TGTGGCCATCCATGCTGACCAGCTGACCCCCACCTGGAGGGTCTACTCC
ACCGGCTCCAATGTCTTCCAAACACGGGCTGGCTGCCTGATTGGCGCTG
AGCATGTGAACAACTCCTATGAGTGTGACATCCCCATTGGCGCTGGCAT
CTGTGCCTCCTACCAAACCCAGACCAACTCCCCTCGGAGGGCTCGCTCT
GTGGCTTCTCAGTCTATCATTGCCTACACCATGTCCCTGGGCGCTGAGA
ACTCTGTGGCCTACTCCAACAACTCCATTGCCATCCCCACCAACTTCAC
CATCTCTGTGACCACCGAGATCCTGCCTGTCTCCATGACCAAGACCTCT
GTGGACTGCACCATGTACATCTGTGGCGACTCCACAGAGTGCTCCAACC
TGCTGCTGCAGTATGGCTCCTTCTGCACCCAACTGAACAGGGCCCTGAC
AGGCATTGCTGTGGAGCAGGACAAGAACACCCAGGAGGTCTTTGCCCAA
GTCAAGCAGATCTACAAGACCCCCCCCATCAAGGACTTTGGCGGCTTCA
ACTTCTCCCAAATCCTGCCTGACCCATCCAAGCCATCCAAGAGGTCCTT
CATTGAGGACCTGCTGTTCAACAAGGTTACCCTGGCTGATGCTGGCTTC
ATCAAGCAGTATGGCGACTGCCTGGGCGACATTGCTGCCAGGGACCTGA
TCTGTGCCCAAAAGTTCAATGGCCTGACAGTGCTGCCCCCCCTGCTGAC
CGATGAGATGATTGCCCAGTACACATCTGCTCTGCTGGCTGGCACAATC
ACCTCTGGCTGGACCTTTGGCGCTGGCGCTGCCCTGCAAATCCCATTTG
CCATGCAAATGGCCTACAGGTTCAATGGCATTGGCGTGACCCAGAACGT
GCTGTATGAGAACCAGAAGCTGATTGCCAACCAGTTCAACTCTGCCATT
AGGCAAGATCCAGGACTCCCTGTCCTCCACCGCCTCTGCCCTGGGCAAG
CTGCAGGATGTGGTGAACCAGAATGCCCAGGCCCTGAACACCCTGGTGA
AGCAGCTGTCCTCCAACTTTGGCGCCATCTCCTCTGTGCTGAATGACAT
CCTGTCCCGGCTGGACAAGGTGGAGGCTGAGGTGCAGATTGACGGCTGA
TCACAGGCAGGCTGCAGTCCCTGCAAACCTATGTGACCCAGCAGCTGAT
CAGGGCTGCTGAGATCAGGGCCTCTGCCAACCTGGCTGCCACCAAGATG
TCTGAATGTGTGCTGGGCCAGTCCAAGAGGGTGGACTTCTGTGGCAAGG
GCTACCATCTGATGTCCTTCCCCCAATCTGCCCCCCATGGCGTGGTCTT
CCTGCATGTGACCTATGTGCCTGCTCAGGAGAAGAACTTCACCACAGCC
CCTGCCATCTGCCATGATGGCAAGGCCCACTTCCCTCGGGAGGGCGTCT
TTGTCTCCAATGGCACCCACTGGTTTGTGACCCAGAGGAACTTCTATGA
GCCTCAGATCATCACCACAGACAACACCTTTGTCTCTGGCAACTGTGAT
GTGGTGATTGGCATTGTGAACAACACAGTCTATGACCCCCTGCAGCCTG
AGCTGGACTCCTTCAAAGAGGAGCTGGACAAGTACTTCAAGAACCACAC
CTCCCCTGATGTGGACCTGGGCGACATCTCTGGCATCAATGCCTCTGTG
GTGAACATCCAGAAGGAGATTGACAGGCTGAATGAGGTGGCCAAGAACC
TGAATGAGTCCCTGATTGACCTGCAAGAGCTGGGCAAGTATGAGCAGTA
CATCAAGTGGCCATGGTACATCTGGCTGGGCTTCATTGCTGGCCTGATT
GCCATTGTGATGGTGACCATCATGCTGTGCTGCATGACCTCCTGCTGCT
CCTGCCTGAAAGGCTGCTGCTCCTGTGGCTCCTGCTGCAAGTTTGATGA
GGATGACTCTGAGCCTGTGCTGAAGGGCGTGAAGCTGCACTACACCTAA
TAA

Hereinafter, the technical solution of the present disclosure will be further illustrated in combination with the experiments.

Construction of Vectors, pVAX-NB1, pVAX-NB2 and pVAX-NB4, for the Expression of the Spike Gene:

Using NB1 and NB2 as templates, respectively, the NB1 fragment was obtained through PCR amplification with primers NB1-F and NB1-R, the NB2 fragment was obtained through PCR amplification with primers NB2-F and NB2-R, and the NB4 fragment was obtained through PCR amplification with primers NB4-F and NB4-R. The vector plasmid backbone pVAX was PCR amplified, by using CMV-R and BGH-F as primers, and using a pVAX plasmid, which was owned by our company, as the template. Then, two-fragment in vitro recombination was carried out by using a homologous recombination enzyme (Exnase) with the NB1, NB2 or NB4 fragment, respectively, to obtain pVAX-NB1, pVAX-NB2 and pVAX-NB4.

Detection of Spike Gene Expression

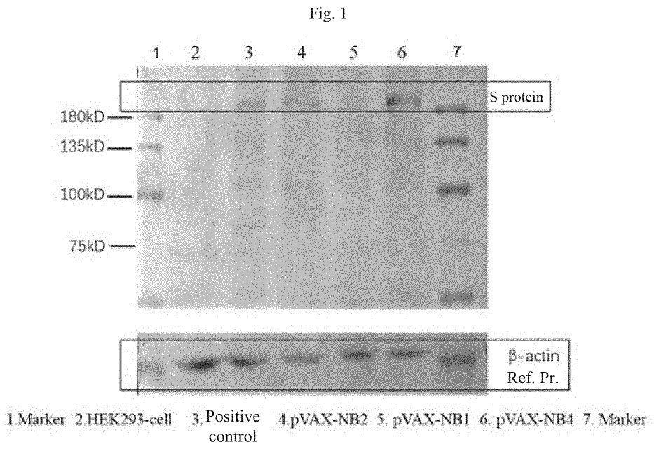

According to a conventional method, transfecting HEK293 cells with 2.5 μg of pVAX-NB1, pVAX-NB2 and pVAX-NB4, respectively, with cationic liposomes for 48 hours, and then collecting the cells. Such samples were processed according to a conventional Western Blot method, and were detected for the protein (FIG. 1). It could be seen that no S protein expression was detected in the pVAX-NB1 sample, while the expression of the S protein could be observed in the codon-optimized pVAX-NB2 and pVAX-NB4 samples. The expression level of S protein for pVAX-NB4 was about 2-3 times of that of S protein for pVAX-NB2. This indicates that the NB4 sequence produced unexpected effect.

Animal Immunogenicity Experiments for DNA Vaccines

Mice were intramuscularly injected with the S protein-expressing plasmids pVAX-NB1 and pVAX-NB2, 100 μg DNA/animal. In particular, 100 μg plasmids were added with 10% glucose to make it up to 200 μl in volume, and added with 10 μl Turbofect (Thermosfisher) according to the manufacturer's instructions. Orbital blood was collected 14 days after the immunization, and the antibody level in the serum was detected by adopting the S protein of the novel coronavirus as the antigen through enzyme-linked immunosorbent assay (ELISA). The specific process was as follows:

1) adding 100 μl of PBS and 50 ng of the S protein per well in a 96-well plate, incubating overnight at 4° C., for 16-18 h;

2) removing the supernatant, washing with PBST once, adding 200 μl of 5% skim milk for blocking at room temperature for 2 h;

3) washing with PBST twice;

4) adding the samples: adding 100 μl of diluted serum samples, incubating at 37° C. for 2 h, and then washing with PBST for 5 times;

5) adding enzyme-labeled antibodies: adding 100 μl of diluted HRP-labeled IgM or IgG secondary antibody, and incubating at 37° C. for 2 h;

6) washing with PBST for 6-8 times;

7) adding substrate solution for development: adding 100 μl of TMB for development;

8) terminating the reaction: adding with 50 μl of 1 M sulfuric acid to terminate the reaction; and

9) determining the results: determining the OD values, and controlling the OD values between 0.1 and 4.

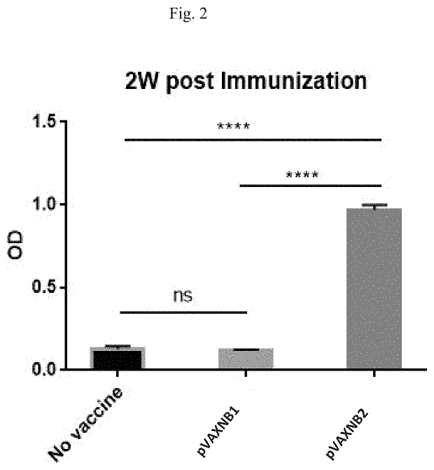

The experimental results showed that, NB2 could induce the generation of antibodies in the mice (FIG. 2).

1. A nucleotide sequence encoding a peptide triggering immunogenicity against Novel Severe Acute Respiratory Syndrome Coronavirus 2 (SARS-CoV-2), comprising at least one selected from:
a) a nucleotide sequence as shown by SEQ ID NO: 1;
b) a nucleotide sequence as shown by SEQ ID NO: 2; or
c) a nucleotide sequence having at least 90% sequence identity with the nucleotide sequence as shown by SEQ ID NO: 1.

2. The nucleotide sequence according to claim 1, wherein the peptide is capable of:
inducing an immune response in a human body;
generating a molecular biology reporter;
generating a molecule for detection;
regulating a gene function; or
functioning as a therapeutic molecule.

3. An expression vector comprising the nucleotide sequence according to claim 1.

4. The expression vector according to claim 3, wherein the vector is a viral vector.

5. The expression vector according to claim 4, wherein the viral vector is an adenovirus vector.

6. (canceled)

7. A nucleotide-based composition comprising the nucleotide sequence according to claim 1 or the expression vector according to claim 3.

8. The nucleotide-based composition according to claim 7, further comprising a pharmaceutically-acceptable adjuvant, carrier, diluent or excipient.

9. (canceled)

10. A method for preventing or treating COVID-19, comprising administering a prophylactically or therapeutically effective amount of the nucleotide-based composition according to claim 7 to a human.

11. The method according to claim 10, wherein the vector is a viral vector.

12. The method according to claim 11, wherein the viral vector is an adenovirus vector.

13. The method according to claim 10, wherein the nucleotide-based composition further comprises a pharmaceutically-acceptable adjuvant, carrier, diluent or excipient.

15. The method according to claim 11, wherein the nucleotide-based composition further comprises a pharmaceutically-acceptable adjuvant, carrier, diluent or excipient.

16. The method according to claim 12, wherein the nucleotide-based composition further comprises a pharmaceutically-acceptable adjuvant, carrier, diluent or excipient.

17. The nucleotide-based composition according to claim 7, wherein the vector is a viral vector.

18. The nucleotide-based composition according to claim 17, wherein the viral vector is an adenovirus vector.

19. The nucleotide-based composition according to claim 17, further comprising a pharmaceutically-acceptable adjuvant, carrier, diluent or excipient.

20. The nucleotide-based composition according to claim 18, further comprising a pharmaceutically-acceptable adjuvant, carrier, diluent or excipient.


SEQUENCE LISTING (2 sequences)

SEQ ID NO: 1 - DNA, 3822 bp, Artificial Sequence
  Optimized nucleotide sequence
atgttcgtgtttctggtgctgctgcctctggtgagctcccagtgcgtgaacctgaccacaaggacccagctgccacctgcctataccaatagcttcacacggggcgtgtactatcccgacaaggtgtttagatctagcgtgctgcactccacccaggatctgtttctgcctttcttttctaacgtgacatggttccacgccatccacgtgtccggcaccaatggcacaaagcggttcgacaatccagtgctgccctttaacgatggcgtgtacttcgcctccaccgagaagtctaacatcatcagaggctggatctttggcaccacactggacagcaagacccagtccctgctgatcgtgaacaatgccacaaacgtggtcatcaaggtgtgcgagttccagttttgtaatgatcccttcctgggcgtgtactatcacaagaacaataagtcttggatggagagcgagtttagggtgtattcctctgccaacaattgcacctttgagtacgtgagccagcctttcctgatggacctggagggcaagcagggcaatttcaagaacctgagggagttcgtgtttaagaatatcgatggctacttcaagatctactccaagcacacaccaatcaacctggtgcgcgacctgccacagggcttctctgccctggagccactggtggatctgcccatcggcatcaacatcacccggtttcagacactgctggccctgcacagaagctacctgaccccaggcgacagctcctctggatggacagcaggagctgccgcctactatgtgggctatctgcagccccgcaccttcctgctgaagtacaacgagaatggcaccatcacagacgcagtggattgcgccctggaccccctgtctgagaccaagtgtacactgaagagctttacagtggagaagggcatctaccagaccagcaacttcagggtgcagccaacagagtccatcgtgcgctttcccaatatcaccaacctgtgcccttttggcgaggtgttcaatgccacacgcttcgccagcgtgtacgcctggaataggaagcgcatctccaactgcgtggccgactattctgtgctgtacaacagcgcctccttctctacctttaagtgttatggcgtgagccccaccaagctgaatgatctgtgctttacaaacgtgtacgccgattccttcgtgatcaggggcgacgaggtgcgccagatcgcaccaggacagaccggcaagatcgcagactacaattataagctgcctgacgatttcacaggctgcgtgatcgcctggaactctaacaatctggatagcaaagtgggcggcaactacaattatctgtaccggctgtttagaaagtctaatctgaagccattcgagcgggacatctccaccgagatctaccaggccggctctacaccctgcaatggcgtggagggctttaactgttatttccctctgcagtcctacggcttccagccaaccaacggcgtgggctatcagccctacagagtggtggtgctgtcttttgagctgctgcacgcacctgcaaccgtgtgcggcccaaagaagagcacaaatctggtgaagaacaagtgcgtgaacttcaacttcaacggactgaccggcacaggcgtgctgaccgagagcaacaagaagttcctgccatttcagcagttcggcagggacatcgcagataccacagacgccgtgcgcgaccctcagaccctggagatcctggacatcacaccatgttccttcggcggcgtgtctgtgatcaccccaggcaccaatacatccaaccaggtggccgtgctgtatcaggacgtgaattgcacagaggtgcccgtggcaatccacgcagatcagctgacccctacatggcgggtgtactctaccggcagcaacgtgttccagacaagagccggatgcctgatcggagcagagcacgtgaacaatagctatgagtgcgacatccctatcggcgccggcatctgtgcctcctaccagacccagacaaactccccaaggagagcccggtctgtggccagccagtccatcatcgcctataccatgagcctgggcgccgagaacagcgtggcctactccaacaattctatcgccatccctaccaacttcacaatcagcgtgaccacagagatcctgccagtgagcatgaccaagacatccgtggactgcaccatgtatatctgtggcgattccacagagtgttctaacctgctgctgcagtacggctccttttgcacccagctgaatagagccctgacaggcatcgccgtggagcaggacaagaacacccaggaggtgttcgcccaggtgaagcagatctacaagacaccacccatcaaggactttggcggcttcaacttcagccagatcctgcccgatcctagcaagccatccaagcggtcttttatcgaggacctgctgttcaacaaggtgaccctggccgatgccggcttcatcaagcagtatggcgattgtctgggcgacatcgccgccagagacctgatctgcgcccagaagtttaatggcctgaccgtgctgcctccactgctgacagatgagatgatcgcacagtacacctctgccctgctggccggcaccatcacaagcggatggacattcggcgcaggagccgccctgcagatcccctttgccatgcagatggcctatcggttcaacggcatcggcgtgacccagaatgtgctgtacgagaaccagaagctgatcgccaatcagtttaacagcgccatcggcaagatccaggactctctgagctccaccgccagcgccctgggcaagctgcaggatgtggtgaatcagaacgcccaggccctgaatacactggtgaagcagctgtctagcaacttcggcgccatctcctctgtgctgaatgacatcctgagccggctggacaaggtggaggcagaggtgcagatcgaccggctgatcaccggcagactgcagtccctgcagacctacgtgacacagcagctgatcagggcagcagagatcagggcctctgccaatctggccgccacaaagatgagcgagtgcgtgctgggacagtccaagagggtggacttttgcggcaagggctatcacctgatgagcttcccacagtccgcccctcacggagtggtgtttctgcacgtgacctacgtgccagcccaggagaagaacttcaccacagcccccgccatctgtcacgatggcaaggcccactttcctagggagggcgtgttcgtgagcaacggcacccactggtttgtgacacagcgcaatttctacgagccacagatcatcaccacagacaataccttcgtgtccggcaactgcgacgtggtcatcggcatcgtgaacaatacagtgtatgatcctctgcagccagagctggactcttttaaggaggagctggataagtacttcaagaatcacaccagccccgacgtggatctgggcgacatctctggcatcaatgccagcgtggtgaacatccagaaggagatcgacagactgaacgaggtggccaagaatctgaacgagagcctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgattccgagcctgtgctgaagggcgtgaagctgcactacacctaa

SEQ ID NO: 2 - DNA, 3825 bp, Artificial Sequence
  Optimized nucleotide sequence
atgtttgtcttcctggtgctgctgcctctggtctcttctcagtgtgtgaacctgaccaccaggacacagctgcctcctgcttacaccaactccttcacacggggcgtctactaccctgacaaggtcttcaggtcctctgtgctgcactccacacaagacctgttcctgccattcttctccaacgtgacctggttccatgccatccatgtctctggcaccaatggcaccaagaggttcgacaaccctgtgctgccattcaacgacggcgtctactttgcctccaccgagaagtccaacatcatcaggggctggatctttggcaccaccctggactccaagacccagtccctgctgattgtgaacaatgccaccaacgtggtgatcaaagtctgcgagttccagttctgcaatgacccattcctgggcgtctactaccacaagaacaacaagtcctggatggagtctgagttcagggtctactcctctgccaacaactgcacctttgaatatgtctcccagccattcctgatggacctggagggcaagcagggcaacttcaagaacctgagggagttcgtcttcaagaacatcgacggctacttcaagatctactccaagcacacacccattaatctcgtccgggatctgcctcagggcttctctgccctggagcccctggtggacctgcccattggcatcaacatcacacgctttcagaccctgctggctctgcaccggtcttacctgacacctggcgactcctcctctggctggacagctggcgctgctgcctactatgtcggctacctgcagcctaggaccttcctgctgaagtacaatgagaatggcaccatcacagatgctgtggactgtgccctggaccccctgtctgagaccaagtgcaccctgaagtccttcacagtcgagaagggcatctaccagacctccaacttccgggtgcagcccacagagtccattgtcaggttccccaacatcaccaacctgtgcccatttggcgaggtcttcaatgccacacgctttgcctctgtctatgcctggaacaggaagaggatctccaactgtgtggctgactactctgtgctgtacaactctgcctccttctccacatttaagtgctatggcgtctcccccaccaagctgaatgacctgtgcttcaccaatgtctatgctgactcctttgtgatccggggcgatgaagttaggcagattgcccctggccagacaggcaagattgctgactacaactacaagctgcctgatgacttcaccggctgtgtgattgcctggaactccaacaacctggactctaaagttggcggcaactacaactacctgtacaggctgttcaggaagtccaacctgaagccatttgagagggacatctccacagagatctaccaggctggctccaccccatgcaatggcgtggagggcttcaactgctacttccccctgcaatcctatggcttccaacccaccaatggcgtgggctaccagccatacagggtggtggtgctgtcctttgagctgctgcatgcccctgccaccgtctgtggccccaagaagtccaccaacctcgtgaagaacaaatgtgtgaacttcaacttcaacggcctgacaggcacaggcgtgctgacagagtccaacaagaagttcctgccattccaacagtttggcagggacattgctgacaccaccgatgctgtcagggacccccagaccctggagatcctggacatcaccccatgctcctttggcggcgtctctgtgatcacccctggcaccaacacctctaatcaagtcgctgtgctgtaccaggacgtgaactgcacagaggttcctgtggccatccatgctgaccagctgacccccacctggagggtctactccaccggctccaatgtcttccaaacacgggctggctgcctgattggcgctgagcatgtgaacaactcctatgagtgtgacatccccattggcgctggcatctgtgcctcctaccaaacccagaccaactcccctcggagggctcgctctgtggcttctcagtctatcattgcctacaccatgtccctgggcgctgagaactctgtggcctactccaacaactccattgccatccccaccaacttcaccatctctgtgaccaccgagatcctgcctgtctccatgaccaagacctctgtggactgcaccatgtacatctgtggcgactccacagagtgctccaacctgctgctgcagtatggctccttctgcacccaactgaacagggccctgacaggcattgctgtggagcaggacaagaacacccaggaggtctttgcccaagtcaagcagatctacaagaccccccccatcaaggactttggcggcttcaacttctcccaaatcctgcctgacccatccaagccatccaagaggtccttcattgaggacctgctgttcaacaaggttaccctggctgatgctggcttcatcaagcagtatggcgactgcctgggcgacattgctgccagggacctgatctgtgcccaaaagttcaatggcctgacagtgctgccccccctgctgaccgatgagatgattgcccagtacacatctgctctgctggctggcacaatcacctctggctggacctttggcgctggcgctgccctgcaaatcccatttgccatgcaaatggcctacaggttcaatggcattggcgtgacccagaacgtgctgtatgagaaccagaagctgattgccaaccagttcaactctgccattggcaagatccaggactccctgtcctccaccgcctctgccctgggcaagctgcaggatgtggtgaaccagaatgcccaggccctgaacaccctggtgaagcagctgtcctccaactttggcgccatctcctctgtgctgaatgacatcctgtcccggctggacaaggtggaggctgaggtgcagattgacaggctgatcacaggcaggctgcagtccctgcaaacctatgtgacccagcagctgatcagggctgctgagatcagggcctctgccaacctggctgccaccaagatgtctgaatgtgtgctgggccagtccaagagggtggacttctgtggcaagggctaccatctgatgtccttcccccaatctgccccccatggcgtggtcttcctgcatgtgacctatgtgcctgctcaggagaagaacttcaccacagcccctgccatctgccatgatggcaaggcccacttccctcgggagggcgtctttgtctccaatggcacccactggtttgtgacccagaggaacttctatgagcctcagatcatcaccacagacaacacctttgtctctggcaactgtgatgtggtgattggcattgtgaacaacacagtctatgaccccctgcagcctgagctggactccttcaaagaggagctggacaagtacttcaagaaccacacctcccctgatgtggacctgggcgacatctctggcatcaatgcctctgtggtgaacatccagaaggagattgacaggctgaatgaggtggccaagaacctgaatgagtccctgattgacctgcaagagctgggcaagtatgagcagtacatcaagtggccatggtacatctggctgggcttcattgctggcctgattgccattgtgatggtgaccatcatgctgtgctgcatgacctcctgctgctcctgcctgaaaggctgctgctcctgtggctcctgctgcaagtttgatgaggatgactctgagcctgtgctgaagggcgtgaagctgcactacacctaataa